(19) United States
(12) Patent Application Publication
(10) Pub. No.: US 2007/0154412 A1
(43) Pub. Date: Jul. 5, 2007

(54) Dentifrices comprising biogenic silica materials

(76) Inventors: Nolan Lee Phillips, Perryville, MD (US); Duen-Wu Hua, Edgewood, MD (US); William C. Fultz, Rising Sun, MD (US); Patrick Donald McGill, Darlington, MD (US)

Correspondence Address: J M HUBER CORPORATION, 333 THORNALL STREET, PATENT DEPARTMENT, EDISON, NJ, 08837-2220, US

(21) Appl. No.: 11/323,662

(22) Filed: Dec. 30, 2005

(51) Int. Cl. A61K 8/97

(52) U.S. Cl. 424/58

(57) ABSTRACT

Unique dentifrices comprising unique abrasive biogenic silica materials are provided. Such compositions exhibit excellent abrasive characteristics, either alone, or in combination with other types of abrasives. In such combinations (with precipitated silica materials, as one example), simultaneously high pellicle film cleaning properties and moderate dentin abrasion levels are possible in order to accord the user a dentifrice that effectively cleans tooth surfaces without detrimentally abrading such surfaces, even at low levels of such biogenic silica additives. Such biogenic silica particles thus surprisingly accord beneficial properties within dentifrice compositions. Encompassed within this invention is the method of utilizing such biogenic silica products within dentifrices, either as the majority abrasive component, or in combination with any other type of commonly used abrasive material.

### FIELD OF THE INVENTION

This invention relates to unique dentifrices comprising unique abrasive biogenic silica materials. Such compositions exhibit excellent abrasive characteristics, either alone, or in combination with other types of abrasives. In such combinations (with precipitated silica materials, as one example), simultaneously high pellicle film cleaning properties and moderate dentin abrasion levels are possible in order to accord the user a dentifrice that effectively cleans tooth surfaces while reducing the abrasion of the dentifrice, even at low levels of such biogenic silica additives. Such biogenic silica particles thus surprisingly accord beneficial properties within dentifrice compositions. Encompassed within this invention is the method of utilizing such biogenic silica products within dentifrices, either as the majority abrasive component, or in combination with any other type of commonly used abrasive material.

### BACKGROUND OF THE PRIOR ART

An abrasive substance has typically been included in conventional dentifrice compositions in order to remove various deposits, including pellicle film, from the surface of teeth. Pellicle film is tightly adherent and often contains brown or yellow materials which impart a discoloration to the teeth. While cleaning is important, the abrasive should not be so aggressive so as to damage the teeth. Ideally, an effective dentifrice abrasive material maximizes pellicle film removal while causing minimal abrasion and damage to the hard tooth tissues. Consequently, among other things, the performance of the dentifrice is highly sensitive to the extent of abrasion caused by the abrasive ingredient. Conventionally, the abrasive cleaning material has been introduced in flowable dry powder form to dentifrice compositions, or via redispersions of flowable dry powder forms of the polishing agent prepared before or at the time of formulating the dentifrice. Also, and more recently, slurry forms of such abrasives have been provided to facilitate storage, transport, and introduction within target dentifrice formulations.

Synthetic low-structure silica products have been utilized for such a purpose due to the effectiveness such materials provide as abrasives, as well as low toxicity characteristics and compatibility with other dentifrice components, such as sodium fluoride, as one example. When preparing synthetic silica products, the objective is to obtain silica products which provide maximal cleaning with minimal impact to the hard tooth surfaces. Dental researchers are continually concerned with identifying abrasive materials that meet such objectives.

Such components must be viable as ingredients within dentifrice compositions in terms of compatibility with active components, ability to exhibit rheological modification in formulations for proper dentifrice form (both functionally and aesthetically by the user), and all while simultaneously present in an amount that is cost-effective and having sufficient abrasive and cleaning performance. Dentifrices and other like paste materials must exhibit proper rheological properties for improved control, such as viscosity build, stand up, brush sag, and the like. For toothpaste formulations, for example, there is a need to provide a stable paste that can meet a number of consumer requirements, including, and without limitation, the ability to be transferred out of a container (such as a tube) via pressure (i.e., squeezing of the tube) as a dimensionally stable paste and to return to its previous state upon removal of such pressure, the ability to be transferred in such a manner to a toothbrush-head easily and without continued flow out of the tube after such transference, the propensity to remain dimensionally stable on the brush prior to use and when applied to target teeth prior to brushing, and the exhibiting of proper mouthfeel for aesthetic purposes, at least, for the benefit of the user.

Generally, dentifrices are comprised of a majority of one or more humectants (such as sorbitol, glycerin, polyethylene glycol, and the like) in order to permit proper suspension and delivery of the oral care product, an abrasive (such as, typically, precipitated silica) for proper mechanical cleaning and polishing of the subject teeth, water, and other active components (such as fluoride-based compounds for anticaries benefits) and other components to provide other function such as foam and sensory appeal. The ability to impart proper rheological benefits to such a dentifrice is accorded through the proper selection and utilization of thickening agents (such as hydrated silicas, hydrocolloids, gums, and the like) to form a proper network of support to properly contain such important humectant, abrasive, and anticaries ingredients.

A number of water-insoluble, abrasive polishing agents have been used or described for dentifrice compositions. These abrasive polishing agents include natural and synthetic abrasive particulate materials. The generally known synthetic abrasive polishing agents include amorphous precipitated silica products and silica gels and precipitated calcium carbonate (PCC). Other abrasive polishing agents for dentifrices have included chalk, magnesium carbonate, dicalcium phosphate and its dihydrate forms, calcium pyrophosphate, zirconium silicate, potassium metaphosphate, magnesium orthophosphate, tricalcium phosphate, perlite, and the like.

Synthetically-produced precipitated low-structure silica products, in particular, have been used as abrasive components in dentifrice formulations due to their cleaning ability, relative safeness, and compatibility with typical dentifrice ingredients, such as humectants, thickening agents, flavoring agents, anticaries agents, and so forth. As known, synthetic precipitated silicas generally are produced by the destabilization and precipitation of amorphous silica from soluble alkaline silicate by the addition of a mineral acid and/or acid gases under conditions in which primary particles initially formed tend to associate with each other to form a plurality of aggregates (i.e., discrete clusters of primary particles), but without agglomeration into a three-dimensional gel structure. The resulting precipitate is separated from the aqueous fraction of the reaction mixture by filtering, washing, and drying procedures, and then the dried product is mechanically comminuted in order to provide a suitable particle size and size distribution.

The silica drying procedures are conventionally accomplished using spray drying, nozzle drying (e.g., tower or fountain), wheel drying, flash drying, rotary wheel drying, oven/fluid bed drying, and the like.

As it is, certain conventional abrasive materials suffer to a certain extent from limitations associated with maximizing cleaning and minimizing dentin abrasion, not to mention complexity in terms of intensive manufacturing procedures, including issues relating to raw material transport, purchase, and ultimate modification. Such raw materials include silica sand and mineral acids (sulfuric, for example), that include their own difficulties in transport, utilization, purification, storage, and ultimate waste disposal. Although such finished abrasive products exhibit excellent dental treatment results, there always exists a general need to develop new types of dental abrasives (and dentifrices thereof) that are less complex to manufacture and/or incorporate within end-use formulations.

Furthermore, the ability to optimize dental abrasion and cleaning characteristics in the past has been limited generally to controlling the structures of the individual precipitated silica components utilized for such purposes. Examples of modifications in precipitated silica structures for such dentifrice purposes are described in the art within such publications as U.S. Pat. Nos. 3,967,563, 3,988,162, 4,420,312, and 4,122,161 to Wason, U.S. Pat. Nos. 4,992,251 and 5,035,879 to Aldcroft et al., U.S. Pat. No. 5,098,695 to Newton et al., and U.S. Pat. Nos. 5,891,421 and 5,419,888 to McGill et al. Modifications in silica gels have also been described within such publications as U.S. Pat. No. 5,647,903 to McGill et al., U.S. Pat. No. 4,303,641, to DeWolf, II et al., U.S. Pat. No. 4,153,680, to Seybert, and U.S. Pat. No. 3,538,230, to Pader et al. Such disclosures teach improvement in such silica materials in order to impart increased pellicle film cleaning capacity and reductions in dentin abrasion levels for dentifrice benefits. However, these typical improvements lack the ability to deliver preferred property levels that accord a dentifrice producer the ability incorporate such an individual material in different amounts with other like components in order to effectuate different resultant levels of such cleaning and abrasion characteristics. To compensate for such limitations, attempts have been undertaken to provide various combinations of silicas to permit targeting of different levels. Such silica combinations involving compositions of differing particle sizes and specific surface areas are disclosed in U.S. Pat. No. 3,577,521. to Karlheinz Scheller et al., U.S. Pat. No. 4,618,488 to Macyarea et al., U.S. Pat. No. 5,124,143 to Muhlemann, and U.S. Pat. No. 4,632,826 to Ploger et al. Such resultant dentifrices, however, fail to provide desired levels of abrasion and high pellicle cleaning simultaneously.

Another attempt has been made to provide physical mixtures of precipitated silicas of certain structures with silica gels, notably within U.S. Pat. No. 5,658,553 to Rice. It is generally accepted that silica gels exhibit edges, and thus theoretically exhibit the ability to abrade surfaces to a greater degree, than precipitated silicas, even low structured types. Thus, the blend of such materials together within this patent provided, at that time, an improvement in terms of controlled but higher levels of abrasiveness coupled with greater pellicle film cleaning ability than precipitated silicas alone. In such a disclosure, it is shown that separately produced and co-incorporated silica gels and precipitated silicas can permit increased PCR and RDA levels but with apparently greater control for lower abrasive characteristics than for previously provided silicas exhibiting very high PCR results. Unfortunately, although these results are certainly a step in the right direction, there is still a largely unfulfilled need to provide a silica-based dental abrasive that exhibits sufficiently high pellicle film cleaning properties with simultaneously lower radioactive dentin abrasive characteristics such that film removal can be accomplished without deleterious dentin destruction. In effect, the need is for a reduced abrasion product that exhibits a significantly higher PCR level versus RDA level than has previously been provided within the dental silica industry. Again, the Rice patent is merely a start toward desirable abrasive characteristics. A manner of providing the benefits of combinations of different forms of physically mixed silicas, but to a very high level of pellicle film cleaning and at a relatively low to moderate degree of dentin abrasion, are thus largely unavailable to the industry at this time. Thus, new possible abrasive silicas for dentifrices that quire less complexity in manufacture, are available as a drop-in component within dentifrices with predictable rheological behavior and/or modification, and exhibits compatibility with other standard dentifrice components, all with excellent results in terms of dental abrasive qualities, could potentially reduce costs within the industry as well as provide improved film cleaning with tailored levels of abrasiveness, would be a particularly useful advancement in the dentifrice industry. To date, however, and again, such an improvement has not been forthcoming.

### ADVANTAGES AND SUMMARY OF THE INVENTION

It has now been found that certain biogenic silicas, namely those derived from rice hulls, can provide highly effective dental abrasion result within dentifrices, either as the sole abrasive component therein, or as a co-additive in combination with other abrasive materials. Of particular advantage is the ability to tailor desired pellicle cleaning (PCR) to radioactive dentin abrasion ratios(RDA) through the combination of particularly selected co-additive abrasive compounds in terms of their general abrasive qualities and their proportion in relation to the amount of rice hull derived silica present within a target dentifrice as well.

In particular, combinations of rice hull derived silica and other dental abrasives (such as precipitated silica, calcium carbonates, and the like) appear to provide potential high levels pellicle film cleaning properties compared with a range of highly desirable lower radioactive dentin abrasion results thus providing the optimization of cleaning while providing a larger margin of abrasion protection to the ultimate user.

It has been realized that the utilization of such rice hull derived silica products within dentifrices provides surprisingly effective abrasion characteristics. In combination with other known dental abrasives, the results are highly unexpected in that such combinations permit effective pellicle film cleaning with simultaneous low levels (though still effective) abrasion. The overall result has been found to provide the potential to hone the pellicle film cleaning and radioactive dentin abrasion characteristics of such overall abrasives. Such an ability meets a certain level of need within the dentifrice industry as the possibility of an abrasive or combination of abrasives that exhibit high pellicle film cleaning (PCR) properties with simultaneously lower radioactive dentin abrasion (RDA) results has been sought after for a long time. At loadings as the abrasive component within a dentifrice of up to about 20% by weight (of all abrasives), there appears to be a plateau of such an increase in these characteristics (up to a ratio very close to 1.0, surprisingly). However, in excess of that amount there may be a significant decrease in this ratio such that above 20% by weight loading in a dentifrice, the ratio seems to decrease to below 0.80 in most situations. Furthermore, where the rice hull derived silica is the sole abrasive present, the ratio decreases even lower, to below 0.71. Such PCR:RDA ratios appear, in each classification, to depend, however, on the cleaning and abrasiveness level of any other abrasives found therein as well.

All parts, percentages and ratios used herein are expressed by weight unless otherwise specified. All documents cited herein are incorporated by reference.

Accordingly, it is one advantage of the present invention to provide a dental abrasive comprised of rice hull derived silica as the sole abrasive for simplicity in formulation and production. Another advantage of this invention is that desired properties of levels of PCR and RDA may be tailored to suit a particular end-use desired result in accordance with the amount of rice hull silica introduced with a selected amount of other abrasive simultaneously present. Also an advantage of this invention is to provide a dentifrice comprising rice hull derived silica-containing abrasive materials wherein the dentifrice exhibits a range of ratios of PCR to RDA dependent upon the amount of such abrasives materials present as well.

Accordingly, this invention encompasses a dentifrice comprising a rice hull silica derived abrasive and optionally including any other dental abrasive component, wherein said dentifrice exhibits a PCR:RDA of at most 0.70; or, alternatively, such a ratio in excess of 0.70 up to 0.80; and as a second alternative a ratio in excess of 0.80.

Generally, synthetic precipitated silicas are prepared by admixing dilute alkali silicate solutions with strong aqueous mineral acids under conditions where aggregation to the sol and gel cannot occur, stirring and then filtering out the precipitated silica. The resulting precipitate is next washed, dried and comminuted to desired size. One such example may be seen in U.S. Pat. No. 5,891,421 to McGill et al.

The preferred biogenic silica material is derived from rice hulls, as is noted within U.S. Pat. No. 6,406,678. The manufacturing process for such silica products is described in full within that patent, which is herein incorporated by reference to that extent. The description itself of such a manufacturing process is thus as follows as provided within that reference:

While the amount of silica contained in rice hulls may vary somewhat due to geographical region where it is grown, and the strain of rice, silica content of rice hulls is generally in the 13-15% range of dry weight. The silica contained in most biogenic material, such as rice hulls, is substantially all of highly desirable amorphous form, but is bound in a biogenic matrix of many other impurities, particularly long chain hydrocarbons such as lignin and cellulose, but including many inorganic minerals such as calcium, magnesium, etc. and compounds thereof. The rice hull silicas involve the necessary separation of the silica from the other impurities found in the biogenic material, primarily the hydrocarbons thereof. Following removal of the hydrocarbons, removal of small quantities of inorganic minerals that remain may be easily substantially removed. The end product is a finely divided white powder of highly pure amorphous silica.

A first, but optional step, of the rice hull silica generation may be cleaning the rice hulls. Typically this will include screening the hulls to remove stalks, clumps of dirt, leaves and other large bodies therefrom and thereafter washing the hulls, with water, in an aqueous based solution containing a surfactant to enhance wet-ability of the hulls. It is believed that washing the hulls with an aqueous based surfactant solution accelerates absorption of oxidizing solution of a following step, as finely dividing the hulls, by shredding, crushing or other conventional means is also believed to do. Therefore, in this production scheme, the hulls are screened, washed with a surfactant solution and finely divided to accelerate the process. It is however noted that these steps are non-essential, highly pure amorphous silica may be extracted from rice hulls without employing these steps, although duration of the following steps may be increased.

Following optional cleaning and division of the rice hulls is the optional step of soaking them in water, which may be at elevated temperature. Such soaking the hulls in water, which may be, and preferably is, at elevated temperature, removes various soluble impurities therefrom and increases porosity of the hulls (making them more susceptible to penetration by oxidizing solution in the following step), and may also effect some beneficial changes in the lignin and cellulose contained in the hulls. It has been observed that soaking rice hulls at near the boiling point of water for 12 or more hours accelerates the subsequent step of reducing the organic materials of the hulls by soaking them in an aqueous based oxidizing solution.

The first essential step of the rice hull silica production scheme is reducing the organic materials of the hulls by soaking them in an aqueous based solution containing an oxidizing solute. This may be accomplished with any number of materials, including many chlorates, perchlorates, nitrates, permanganates and certain peroxide compounds (such as Fenton's reagent) while comprehended by the invention, although they are not preferred. Peracetic acid is a preferred oxidizing solute because its residue is easily removed in the final, optional, step of the process. However hydrogen peroxide is the most preferred oxidizer because after it is spent water is its only remainder. If the peroxide is not completely spent in processing the hulls, as will typically be the case, so as to ensure full reduction of the organic material of the hulls, the remaining oxygen spontaneously evolves over a short period of time, which evolution may be accelerated by heating, mechanical agitation, electrolytic or various other known means. Accordingly the process disclosed herein is one that is very environmentally friendly.

The initial dosage of hydrogen peroxide (contained in an aqueous solution) of the preferred embodiment of the invention contains approximately 0.1 mole of hydrogen peroxide (about 3.4 grams of peroxide) per kilogram of hulls. It is noted that increasing the temperature of such solution speeds the effect it has on the hulls. Maintaining the temperature of said solution in the 90-100° C. range, over a course of 6-8 hours, has been found fully effective. Using a temperature in excess of 100° C. will require the use of a pressure vessel. While reduction is possible to at least room temperature or below, it is noted that decreased temperature tends to increase time required for reduction exponentially thus, while comprehended, is not preferred. Initial dosage of hydrogen peroxide may be substantially less, so long as during reduction monitoring is had to insure that at least some non-reacted peroxide remains in solution for a sufficient period of time to accomplish desired reduction of the organic materials of the hulls.

Following the above described reducing step the hulls may be thoroughly rinsed with water and are preferably then dried to a water content of 10% or less water content by weight. Rinsing the hulls, if done, should be done with as pure a water as is practical, such as de-ionized or even distilled water, with very low iron or heavy metal content, lest the rinse water itself contribute undesirable impurities to the silica.

Drying can be done by any conventional means, but drying with heated air is preferred since the process herein disclosed creates a readily available source of heat. Following the step of reducing the organic materials of the hulls, and preferably rinsing and drying as described above, the hulls are next “burned” (combusted, or oxidized, by heat in the presence of an oxygenated gas). The preferred temperature range at which the hulls are burned is from about 500-950° C. At temperatures substantially below that range the carbonaceous impurities of the hulls take an excessive length of time to oxidize fully, and at some point may not oxidize at all. At temperatures substantially above that range there is increasing risk that hot spots which will occur due to local exothermic oxidation of impurities, particularly carbonaceous impurities, will begin transforming some of the silica from amorphous to crystalline form, which is not desired.

The hulls are oxidized by elevated temperature, as described above, in the presence of an oxygen containing gas. In order to ensure good oxygenation of all the hulls they are typically placed in a thin bed and air flowed upwardly therethrough. Oxidation of the hulls occurs so rapidly in air at approximately 600° C. that by the time the hulls reach temperature set point, oxidation to a fine, white, amorphous silica, without visually detectable carbon residue, is completed.

Due to the fact that silica is stable, quite porous and insoluble in water and acids (except hydrogen fluoride), it can be further washed, rinsed, flushed with wide variety of acids and other solutions designed to remove particular impurities, such as calcium compounds, which remain following oxidation.

The inventive rice hull derived silica abrasive compositions are ready-to-use additives in the preparation of oral cleaning compositions, such as dentifrices, toothpastes, and the like, particularly suited as a raw material in a toothpaste making process. If combined with other abrasives (such as any of the products offered by J. M. Huber Corporation under the tradename ZEODENT®), such an abrasive may be added in any amount, but generally for higher PCR:RDA ratios (in excess of 0.80), the amount is at most 20% by weight of the total amount of abrasive present, whereas lower ratios of such characteristics (greater than 0.70 up to 0.80), the amount is in excess of 20% and up to 50% by weight, and for less than a 0.70 ratio, the amount is in excess of 50% by weight of the rice hull derived silica.

The inventive rice hull derived silica abrasive compositions may be utilized alone as the cleaning agent component provided in the dentifrice compositions of this invention, although, at least for the high cleaning category materials, the moderately high RDA levels may be unacceptable to some consumers. Thus, a combination of the inventive composite materials with other abrasives physically blended therewith within a suitable dentifrice formulation is potentially preferred in this regard in order to accord targeted dental cleaning and abrasion results at a desired protective level. Thus, any number of other conventional types of abrasive additives may be present within inventive dentifrices in accordance with this invention. Other such abrasive particles include, for example, and without limitation, precipitated calcium carbonate (PCC), ground calcium carbonate (GCC), dicalcium phosphate or its dihydrate forms, silica gel (and of any structure), amorphous precipitated silica (by itself, and of any structure as well), perlite, titanium dioxide, calcium pyrophosphate, hydrated alumina, calcined alumina, insoluble sodium metaphosphate, insoluble potassium metaphosphate, insoluble magnesium carbonate, zirconium silicate, aluminum silicate, and so forth, can be introduced within the desired abrasive compositions to tailor the polishing characteristics of the target formulation (dentifrices, for example, etc.), if desired, as well.

The abrasives (be they the rice hull silica alone or a combination of rice hull silica with any other abrasive material, as described above), when incorporated into dentifrice compositions, are present at a level of from about 5% to about 50% by weight, more preferably from about 10% to about 35% by weight, particularly when the dentifrice is a toothpaste. Overall dentifrice or oral cleaning formulations incorporating the abrasive compositions of this invention conveniently can comprise the following possible ingredients and relative amounts thereof (all amounts in wt % ):

Dentifrice Formulation

| Ingredient | |
|---|---|
| Liquid Vehicle: | Amount |
| humectant(s) (total) | 5–70 |
| deionized water | 5–70 |
| binder(s) | 0.5–2.0 |
| anticaries agent | 0.1–2.0 |
| chelating agent(s) | 0.4–10 |
| silica thickener* | 3–15 |
| surfactant(s) | 0.5–2.5 |
| abrasive | 10–50 |
| sweetening agent | <1.0 |
| coloring agents | <1.0 |
| flavoring agent | <5.0 |
| preservative | <0.5 |

In addition, as noted above, the inventive abrasive could be used in conjunction with other abrasive materials, such as precipitated silica, silica gel, dicalcium phosphate, dicalicum phosphate dihydrate, calcium metasilicate, calcium pyrophosphate, alumina, calcined alumina, aluminum silicate, precipitated and ground calcium carbonate, chalk, bentonite, particulate thermosetting resins and other suitable abrasive materials known to a person of ordinary skill in the art.

In addition to the abrasive component, the dentifrice may also contain one or more organoleptic enhancing agents. Organoleptic enhancing agents include humectants, sweeteners, surfactants, flavorants, colorants and thickening agents, (also sometimes known as binders, gums, or stabilizing agents), Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds. Typical levels of humectants are from about 20 wt % to about 30 wt % of a toothpaste composition.

Sweeteners may be added to the toothpaste composition to impart a pleasing taste to the product. Suitable sweeteners include saccharin (as sodium, potassium or calcium saccharin), cyclamate (as a sodium, potassium or calcium salt), acesulfane-K, thaumatin, neohisperidin dihydrochalcone, ammoniated glycyrrhizin, dextrose, levulose, sucrose, mannose, and glucose.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate,, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the product. Suitable colorants are selected from colorants approved by appropriate regulatory bodies such as the FDA and those listed in the European Food and Pharmaceutical Directives and include pigments, such as TiO₂, and colors such as FD&C and D&C dyes.

Thickening agents are useful in the dentifrice compositions of the present invention to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; as well as mixtures of these compounds. Typical levels of thickening agents or binders are from about 0 wt % to about 15 wt % of a toothpaste composition.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates, such as ; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may also be optionally added to the compositions of the present invention to prevent bacterial growth. Suitable preservatives approved for use in oral compositions such as methylparaben, propylparaben and sodium benzoate, or combinations thereof, may be added in safe and effective amounts.

The dentifrices disclosed herein may also a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like Water provides the balance of the composition in addition to the additives mentioned. The water is preferably deionized and free of impurities. The dentifrice will usually comprise from about 0 to about 60 wt % of water, with some having narrower ranges (from all sources) of from about 5 to about 35%, and others may have even narrower ranges of between 20 wt % to about 35 wt %.

Useful silica thickeners for utilization within such a toothpaste formulation include, as a non-limiting example, an amorphous precipitated silica such as ZEODENT® 165 silica. Other preferred (though non-limiting) silica thickeners are ZEODENT® 163 and/or 167 and ZEOFREE®153, 177, and/or 265 silicas, all available from J. M. Huber Corporation, Havre de Grace Md., U.S.A.

For purposes of this invention, a “dentifrice” has the meaning defined in Oral Hygiene Products and Practice, Morton Pader, Consumer Science and Technology Series, Vol. 6, Marcel Dekker, NY 1988, p. 200, which is incorporated herein by reference. Namely, a “dentifrice” is “ . . . a substance used with a toothbrush to clean the accessible surfaces of the teeth. Dentifrices are primarily composed of water, detergent, humectant, binder, flavoring agents, and a finely powdered abrasive as the principal ingredient . . . a dentifrice is considered to be an abrasive-containing dosage form for delivering anti-caries agents to the teeth.” Dentifrice formulations contain ingredients which must be dissolved prior to incorporation into the dentifrice formulation (e.g. anti-caries agents such as sodium fluoride, sodium phosphates, flavoring agents such as saccharin).

The various silica and toothpaste (dentifrice) properties described herein were measured as follows, unless indicated otherwise.

Median particle size is determined using a Model LA-300 laser light scattering instrument available from Horiba Instruments, Boothwyn, Pa.

To measure brightness, fine powder materials pressed into a smooth surfaced pellet were evaluated using a Technidyne Brightmeter S-5/BC. This instrument has a dual beam optical system where the sample is illuminated at an angle of 45°, and the reflected light viewed at 0°. It conforms to TAPPI test methods T452 and T646, and ASTM Standard D985. Powdered materials are pressed to about a 1 cm thick pellet with enough pressure to give a pellet surface that is smooth and flat and without loose particles or gloss.

The Brass Einlehner (BE) Abrasion test used to measure the hardness of the precipitated silicas/silica gels reported in this application is described in detail in U.S. Pat. No. 6,616,916, incorporated herein by reference, involves an Einlehner AT-1000 Abrader generally used as follows: (1) a Fourdrinier brass wire screen is weighed and exposed to the action of a 10% aqueous silica suspension for a fixed length of time; (2) the amount of abrasion is then determined as milligrams brass lost from the Fourdrinier wire screen per 100,000 revolutions. The result, measured in units of mg loss, can be characterized as the 10% brass Einlehner (BE) abrasion value.

The Radioactive Dentin Abrasion (RDA) values of dentifrices containing the silica compositions used in this invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, which publications and patents are incorporated herein by reference.

The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain with Dentifrice” G. K. Stookey, et al., J. Dental Res., 61, 1236-9, 1982. Both PCR and RDA results vary depending upon the nature and concentration of the components of the dentifrice composition. PCR and RDA values are unitless.

### PREFERRED EMBODIMENTS OF THE INVENTION

### Utilization of Biogenic Silica as Dentifrice Abrasives

### EXAMPLES 1-4

In these examples, several samples of STRATOSIL™ S-100 silica, which is derived from rice hulls, was tested for various properties according to the methods described above and the results are summarized in Table 1.

TABLE 1

Example

| | 1 | 2 | 3 | 4 |
|---|---|---|---|---|
| MPS, μm | 48 | 18.52 | 5.65 | 4.0 |
| Brightness | — | 78.4 | 89.1 | 90.7 |
| Einlehner Abrasion, mg | 20.41 | 40.5 | 25.14 | 20.29 |
| loss | | | | |
| % 325 residue | — | — | — | 0 |
| BET surface area, m²/g | — | — | — | 297 |
| CTAB surface area, m²/g | — | — | — | 123 |
| Oil Absorption | — | — | — | 77 |
| 5% pH | — | — | — | 4.3 |
| Total Pore volume, ml/g | — | — | — | 1.28 |

STRATOSIL™ S-100 silica is derived from rice hulls and is available from International Silica Technologies, LLC, The Woodlands, Tex. Example 1 was obtained as an unmilled, spray dried sample of STRATOSIL S-100 as is demonstrated by its large particle size. Examples 2-4 were obtained as milled samples of STRATOSIL S-100. The very small particle size samples still had a very high Einlehner abrasion value of about 20-25 mg loss, compared to precipitated silica abrasives which typically have an Einlehner abrasion of about 3-8 mg loss.

Toothpaste formulations were prepared using several of the above-described silica examples to demonstrate the optimum dental protection benefits.

To prepare the dentifrices, the glycerin, sodium carboxymethyl cellulose, polyethylene glycol and sorbitol were mixed together and stirred until the ingredients were dissolved to form a first admixture. The deionized water, sodium fluoride, tetrasodium pyrophosphate and sodium saccharin were also mixed together and stirred until these ingredients are dissolved to form a second admixture. These two admixtures were then combined with stirring. Thereafter, the optional color was added with stirring to obtain a “pre-mix”. The pre-mix was placed in a Ross mixer (Model 130 LDM) and silica thickener, inventive abrasive silica and titanium dioxide were mixed in without vacuum. A 30-inch vacuum was drawn and the resultant admixture was stirred for approximately 15 minutes. Lastly, sodium lauryl sulfate and flavor were added and the admixture was stirred for approximately 5 minutes at a reduced mixing speed. The resultant dentifrice was transferred to plastic laminate toothpaste tubes and stored for future testing. The dentifrice formulations are given in Table 2 below. The dentifrice formulation utilized was considered a suitable test dentifrice formulation for the purposes of determining PCR and RDA measurements for the inventive cleaning abrasives.

TABLE 2
Dentifrice Formulations

| | 1 | 2 | 3 | 4 | 5 |
|---|---|---|---|---|---|
| Glycerin, 99.5%, g | 0 | 0 | 0 | 0 | 11.000 |
| Sorbitol, 70%, g | 58.467 | 58.467 | 58.467 | 58.467 | 40.007 |
| Deionized Water, g | 12.715 | 12.715 | 12.715 | 12.715 | 20.000 |
| CARBOWAX ® 600, g | 5.000 | 5.000 | 5.000 | 5.000 | 3.000 |
| CEKOL ® 2000 CMC, g | 0.500 | 0.500 | 0.500 | 0.500 | 1.200 |
| Sodium benzoate, g | 0.500 | 0.500 | 0.500 | 0.500 | 0 |
| Tetrasodium | 0 | 0 | 0 | 0 | 0.500 |
| Pyrophosphate, g | | | | | |
| Sodium Saccharin, g | 0.200 | 0.200 | 0.200 | 0.200 | 0.200 |
| Sodium Fluoride, g | 0.243 | 0.243 | 0.243 | 0.243 | 0.243 |
| ZEODENT ® 165 | 8.500 | 13.500 | 8.500 | 8.500 | 1.500 |
| Silica Thickener, g | | | | | |
| Example 1 silica, g | 10 | 0 | 0 | 0 | 0 |
| Example 2 silica, g | 0 | 5.0 | 0 | 0 | 0 |
| Example 3 silica, g | 0 | 0 | 10 | 0 | 0 |
| Example 4 silica, g | 0 | 0 | 0 | 10 | 20 |
| TiO₂, g | 0.500 | 0.500 | 0.500 | 0.500 | 0.500 |
| Sodium Lauryl Sulfate, g | 1.875 | 1.875 | 1.875 | 1.875 | 1.200 |
| Flavor, g | 1.500 | 1.500 | 1.500 | 1.500 | 0.650 |

ZEODENT ® 165 is an amorphous, precipitated high structure silica thickening agent available from J.M. Huber Corporation, Havre de Grace, Maryland;
CARBOWAX ® 600 is a polyethylene glycol available from the Dow Chemical Company, Midland, Michigan; and
CEKOL ® 2000 is a CMC available from the Noviant Group, Arnhem, the Netherlands.

The dentifrice formulations prepared above were evaluated for PCR and RDA properties, according to the methods described above; the measurements for each dentifrice formulation are provided in Table 3 below.

TABLE 3

| Formulation | RDA | PCR | PCR/RDA |
|---|---|---|---|
| 1 | 133 | 66 | 0.50 |
| 2 | 125 | 75 | 0.60 |
| 3 | 125 | 89 | 0.71 |
| 4 | 138 | 91 | 0.66 |
| 5 | 136 | 107 | 0.79 |

Surprisingly, the RDA values are independent of the silica particle size, essentially having about the same RDA for particles between 48 μm and 4 μm. Also the particle hardness of the STRATOSIL silica, demonstrated by the Einlehner Abrasion value, is not correlated to toothpaste RDA and the RDA is independent of the silica loading level in the toothpaste. However, the PCR values and the PCR/RDA ratio tend to increase as the silica particle size decreases as well as with increased loading of this rice hull silica derived material therein.

### Combinations of Rice Hull Derived Silica and Other Dental Abrasives

### EXAMPLES 5-12

Several examples of blends of amorphous precipitated silica and Example 4 rice hull silica were made weighing the quantities of components given in Table 4 into a plastic sample bag and mixing the silicas together by inverting the closed bag several times until the mixture was homogenous. Two commercial precipitated silica product samples were used for combinations with the rice hull derived silica from above. These products exhibited the following characteristics:

TABLE 5

Commercial Precipitated
Silica Example

| | A | B |
|---|---|---|
| MPS, μm | 12 | 11 |
| Brightness | 97 | 97 |
| Einlehner Abrasion, mg loss | 2.5 | 5.5 |
| % 325 residue | 0.75 | 0.75 |
| % H₂O | 8 | 6 |
| % Na₂SO₄ (by conductivity) | 1 | 1 |
| Oil Absorption | 100 | 88 |
| 5% pH | 7.3 | 7.7 |

TABLE 4

| | Example | Commercial | Commercial |
|---|---|---|---|
| Example | 4 g | Product B g | Product A G |
| 5 | 225 | 75 | 0 |
| 6 | 150 | 150 | 0 |
| 7 | 75 | 225 | 0 |
| 8 | 225 | 0 | 75 |
| 9 | 150 | 0 | 150 |
| 10 | 75 | 0 | 225 |
| 11 | 75 | 0 | 225 |
| 12 | 30 | 0 | 270 |

The silica blend examples described above were incorporated into dentifrice formulations according to the method described in Example 1. The dentifrice formulations are given in Table 5 below.

TABLE 5
Dentifrice Formulations

| | 6 | 7 | 8 | 9 | 10 | 11 | 12 | 13 |
|---|---|---|---|---|---|---|---|---|
| Glycerin, 99.5%, g | 11.000 | 11.000 | 11.000 | 11.000 | 11.000 | 11.000 | 11.000 | 11.000 |
| Sorbitol, 70%, g | 40.007 | 40.007 | 40.007 | 40.007 | 40.007 | 40.007 | 40.007 | 40.007 |
| Deionized Water, g | 20.000 | 20.000 | 20.000 | 20.000 | 20.000 | 20.000 | 20.000 | 20.000 |
| CARBOWAX ® 600, g | 3.000 | 3.000 | 3.000 | 3.000 | 3.000 | 3.000 | 3.000 | 3.000 |
| CEKOL ® 2000 CMC, g | 1.200 | 1.200 | 1.200 | 1.200 | 1.200 | 1.200 | 1.200 | 1.200 |
| Tetrasodium | 0.500 | 0.500 | 0.500 | 0.500 | 0.500 | 0.500 | 0.500 | 0.500 |
| Pyrophosphate, g | | | | | | | | |
| Sodium Saccharin, g | 0.200 | 0.200 | 0.200 | 0.200 | 0.200 | 0.200 | 0.200 | 0.200 |
| Sodium Fluoride, g | 0.243 | 0.243 | 0.243 | 0.243 | 0.243 | 0.243 | 0.243 | 0.243 |
| ZEODENT ® 165 | 1.500 | 1.500 | 1.500 | 1.500 | 1.500 | 1.500 | 1.500 | 1.500 |
| Silica Thickener, g | | | | | | | | |
| Example 5 silica, g | 20 | 0 | 0 | 0 | 0 | 0 | 0 | 0 |
| Example 6 silica, g | 0 | 20 | 0 | 0 | 0 | 0 | 0 | 0 |
| Example 7 silica, g | 0 | 0 | 20 | 0 | 0 | 0 | 0 | 0 |
| Example 8 silica, g | 0 | 0 | 0 | 20 | 0 | 0 | 0 | 0 |
| Example 9 silica, g | 0 | 0 | 0 | 0 | 20 | 0 | 0 | 0 |
| Example 10 silica, g | 0 | 0 | 0 | 0 | 0 | 20 | 0 | 0 |
| Example 11 silica, g | 0 | 0 | 0 | 0 | 0 | 0 | 20 | 0 |
| Example 12 silica, g | 0 | 0 | 0 | 0 | 0 | 0 | 0 | 20 |
| TiO₂, g | 0.500 | 0.500 | 0.500 | 0.500 | 0.500 | 0.500 | 0.500 | 0.500 |
| Sodium Lauryl Sulfate, g | 1.200 | 1.200 | 1.200 | 1.200 | 1.200 | 1.200 | 1.200 | 1.200 |
| Flavor, g | 0.650 | 0.650 | 0.650 | 0.650 | 0.650 | 0.650 | 0.650 | 0.650 |

The dentifrice formulations prepared above were evaluated for PCR and RDA properties, according to the methods described above; the measurements for each dentifrice formulation are provided in Table 6 below.

TABLE 6

| Formulation | | | |
|---|---|---|---|
| No. | RDA | PCR | PCR/RDA |
| 6 | 136 | 100 | 0.74 |
| 7 | 132 | 100 | 0.76 |
| 8 | 126 | 97 | 0.77 |
| 9 | 132 | 101 | 0.77 |
| 10 | 128 | 99 | 0.77 |
| 11 | 112 | 96 | 0.86 |
| 12 | 114 | 96 | 0.84 |
| 13 | 96 | 91 | 0.95 |

ZEODENT 114 is a less abrasive silica, than ZEODENT 119 silica, demonstrated by their Einlehner values. Toothpaste Formulations 6-8 contained blends of STRATOSIL rice hull silica and ZEODENT 119 silica in the same ratios as Toothpaste Formulations 9-11 containing the rice hull silica and ZEODENT 114 silica. For the higher blend ratios of precipitated silica to rice hull silica (Formulation 11 verses Formulation 8), a higher PCR/RDA ratio is obtained when the less abrasive ZEODENT 114 was used. It is possible to obtain toothpaste that has a PCR/RDA ratio approaching 1 by varying the ratio of STRATOSIL silica to conventional silica as is seen in Formulation 13. The appropriate abrasiveness of the conventional silica which is blended with the rice hull silica provides dentifrice with higher PCR/RDA ratios.

While the invention will be described and disclosed in connection with certain preferred embodiments and practices, it is in no way intended to limit the invention to those specific embodiments, rather it is intended to cover equivalent structures structural equivalents and all alternative embodiments and modifications as may be defined by the scope of the appended claims and equivalence thereto.

1. A dentifrice comprising a rice hull derived silica abrasive and optionally including any other dental abrasive component, wherein said dentifrice exhibits a PCR:RDA in excess of 0.70 up to 0.80.

2. The dentifrice of claim 1 wherein another dental abrasive component is present.

3. The dentifrice of claim 2 wherein said other dental abrasive component is selected from the group consisting of precipitated silica products, silica gels, precipitated calcium carbonate, ground calcium carbonate, chalk, magnesium carbonate, dicalcium phosphate and its dihydrate forms, calcium pyrophosphate, zirconium silicate, potassium metaphosphate, magnesium orthophosphate, tricalcium phosphate, perlite, and any mixtures thereof.

4. The dentifrice of claim 3 wherein said other dental abrasive component is a precipitated silica product.

5. The dentifrice of claim 2 wherein the total amount of abrasives present within said dentifrice is up to 30% by weight therein, and wherein said rice hull derived silica is present in an amount of up to 25% by weight therein.

6. The dentifrice of claim 3 wherein the total amount of abrasives present within said dentifrice is up to 30% by weight therein, and wherein said rice hull derived silica is present in an amount of up to 25% by weight therein.

7. The dentifrice of claim 4 wherein the total amount of abrasives present within said dentifrice is up to 30% by weight therein, and wherein said rice hull derived silica is present in an amount of up to 25% by weight therein.
